(19) United States
(12) Patent Application Publication
(10) Pub. No.: US 2023/0088719 A1
(43) Pub. Date: Mar. 23, 2023

(54) USE OF BMP INHIBITORS IN THE TREATMENT OF MOLAR PREGNANCY

(71) Applicant: BOGAZICI UNIVERSITESI, Besiktas/Istanbul (TR)

(72) Inventors: Nesrin OZOREN, Besiktas/Istanbul (TR); Aybuke GARIPCAN, Besiktas/Istanbul (TR)

(21) Appl. No.: 17/798,143

(22) PCT Filed: Feb. 10, 2021
(86) PCT No.: PCT/TR2021/050115

§ 371 Date: Aug. 8, 2022

(30) Foreign Application Priority Data
Feb. 10, 2020 (TR) 2020/01995

(51) Int. Cl. A61K 31/519; A61K 31/4418; A61K 45/06; A61P 15/02

(52) U.S. Cl. CPC A61K 31/519; A61K 31/4418; A61K 45/06; A61P 15/02

(57) ABSTRACT

The present invention describes a new method for the prevention or treatment of molar pregnancies, more particularly familial recurrent hydatidiform mole, and more particularly hydatidiform mole diseases caused by a mutation in the NLRP7 gene.

### TECHNICAL FIELD

The invention relates to the use of BMP inhibitors in the treatment of molar pregnancies (molar hydatiform or hydatid mole), more particularly molar pregnancies caused by the NLRP7 mutation.

### STATE OF THE ART

Molar pregnancy is a gestational trophoblastic disease in which the baby never grows or grows abnormally. Here, the trophoblast cells forming the placenta show hyper proliferation (abnormal trophoblastic proliferation) and grow. Uncontrolled growth causes a development of a grape cluster-like structure. Here, embryo formation can be observed, and it is also possible that no embryos are formed. Molar pregnancies are also known as hydatidiform mole due to the abnormal structure of the placenta.

Molar pregnancy is divided into two subtypes; complete mole and partial mole. While the embryo does not form in complete molar (CM) pregnancies, the placental cells develop abnormally and a plurality of cyst structures occurs. It usually occurs when an enucleate ovum is fertilized by one or two sperms, and thus the fertilized ovum contains only the chromosomes from the father. Accordingly, the diploid karyotype in which both chromosome sets come from the father can be seen in the abovementioned complete molar pregnancy types.

In partial molar (PM) pregnancy, biparentaral triploid karyotype is observed along with an abnormally developing placenta. However, an improper fetus formation can be observed. In partial molar pregnancies, the ovum is normal, it contains 23 chromosomes as it should be. However, as a result of fertilization, the genetic material of the two sperms combines with the genetic material carried by the ovum and the embryo carries more chromosomes than it should be. For this reason, the fetus cannot continue its development and the pregnancy ends with an early abortus.

Complete mole pregnancies are more likely to transform into gestational trophoblast tumors (5-25%) than partial molar pregnancies.

Diagnosis of molar pregnancy is easily carried out by ultrasonography. Instead of the sac, amniotic fluid, and placenta on ultrasound images, the abovementioned hydatid structure is seen.

In the state of the art, the treatment of molar pregnancy involves cleaning the uterus with an operation similar to an abortion. This operation is riskier than a typical abortion procedure. In some cases, it may be necessary to remove the uterus completely. After the operation, if the Beta hCG value which exceeds 100.000 in molar pregnancy does not decrease, chemotherapy treatment may be needed. After said treatment, it is possible for the patient to be pregnant again only after a certain waiting period.

The frequency of repeating molar pregnancy for the second time is approximately 1%. It is very unlikely that it will repeat for the third time. (Ulker V. et al. (2013) European Journal of Obstetrics Gynecology and Reproductive Biology 170(1):188-192).

In case of continuous recurrence of molar pregnancies, the familial recurrent molar hydatidiform (Familial Recurrent Hydatidiform Mole (FRHM)), which is a very rare disease, is considered. A maternal effect gene, NLRP7 has been identified as a first causative gene in FRHM patients. It is not possible for FRHM patients carrying NLRP7 mutations to have a baby. These patients can only have a child by having a healthy pregnancy with ovum donation. Considering that said molar pregnancies are caused by maternal mutation, it is obvious that there is a need for a treatment method in the technical field with the acceptance that it will almost always recur.

In previous studies, in the prior art, it has been identified that the mutations associated with molar pregnancy are in the NLRP7 gene in a very significant part of FRHM patients.

NLRP7 is one of the members of the NLR (Node Family Receivers) family. The human NLRP7 gene is located at locus 19q13.4. All of its isoforms have the characteristic domains of the NLR family. These are the PYD (pyrin domain), NACHT (NAIP, CIITA; area available in HET-E, TP-1), NAD (NACHT domain) and LRR (Leucine Rich Repeat domain).

Previously, 48 different mutations in the NLRP7 gene were reported (Akoury et al. (2015) Reprod Biomed Online 31:120-4). These mutations are found in all regions of NLRP7 gene. Both what is known about the structure and function of protein and NLRP7 mutation studies related to molar pregnancy show that even a little change in the structure of the protein leads to a loss of function, and thus any mutation in the gene can cause molar pregnancy.

These mutations in NLRP7 are seen in 48 to 80% of recurrent molar pregnancies. Studies carried out with molar pregnancy patients showed that these mutations can be s small deletions or insertions (less than 20 bp), large deletions or insertions, base changes, and complex rearrangements. In addition to these mutations, insufficient protein production due to early stop codons ands the frame shifts was also observed. Mutations in the maternal effect gene, NLRP7, are particularly important in patients with complete molar and have been found to be directly related to the disease.

In recent years, Mahadevan et al. have presented a non-inflammatory role for NLRP7. Accordingly, NLRP7 plays a role in differentiation of human embryonic stem cells into trophoblast cells. Therefore, the decrease in the NLRP7 expression levels causes changes in DNA methylation and this change causes differentiation of human embryonic stem cells into trophoblasts. (Mahadevan et. Al (2014) Hum Mol Genet 23 (3): 706-716).

Since molar pregnancy occurs due to hyper proliferation of trophoblast cells, understanding the trophoblast formation mechanism is important to understand the causes of this disease. As it is known, trophoblast cells are the first cells to differentiate from the fertilized ovum (zygote) and form a large part of the placenta. The factors on the differentiation of human embryonic cells into trophoblasts can be determined by some studies in vitro. However, serious limitations are discussed in the state of the art for in vivo studies.

Further, it has been described in the state of the art that overexpression of NLRP7 has an effect on endometrial cancer, embryonal carcinoma and testicular seminoma. (Ohno, Kinoshita et al. (2008) Anticancer Research 25(4C): 2493-2497) Also, it is assumed that it has a possible role in the proliferation of germline cells.

In the rodent genome, NLRP7 gene is absent. (Radian, de Almeida et al. 2013). For this reason, it is not possible to conduct research on FRHM caused by the NLRP7 mutations by using mice models, which are model organisms frequently used in molecular biology studies. Studying on human embryos is also not possible due to ethical considerations.

By means of the developments in recent years, some studies that need to be carried out on human embryos can be performed on induced pluripotent stem cells developed as an alternative for embryo cells. Induced pluripotent stem cells (iPSC) are pluripotent cells obtained by gene transfer of Yamanaka factors (named OCT3/4, SOX2, KLF4, c-MYC) into a somatic cell in order to have properties similar to embryonic stem cells. Pluripotent stem cells can give rise to 3 germ layers (ectoderm, mesoderm, endoderm) of the organism and is normally only present in embryonic stem cells. Since they are similar to embryonic stem cells, tests that cannot be performed with embryonic stem cells for ethical reasons can be performed on iPSCs. iPSCs can be generated from any patients by using Yamanaka factors and differentiated into many cell types.

Therefore, iPSCs present a unique platform where cells mimic the phenotype of the diseases. In studies carried out previously, iPSC disease models are used in revealing the disease mechanism, showing drug effects, developing new therapeutic agents and patient specific cell therapies.

However, despite these developments, the role of the NLRP7 gene in complete molar pregnancies has not been clarified and a treatment method has not been developed. The studies carried out up to today have not revealed the underlying mechanism behind FRHM except showing a connection between complete molar pregnancy and the NLRP7 mutations, and have not suggested any treatment method.

Thus, in the technique, there is a need for methods that provide effective treatment of complete molar pregnancies and particularly familial recurrent complete molar pregnancies.

### SUMMARY OF THE INVENTION

The main object of the present invention is to provide prevention or treatment of molar pregnancies, particularly familial recurrent hydatidiform mole (FRHM).

An object of the present invention is to develop a new method for the prevention or treatment of complete molar pregnancies, more particularly molar pregnancies caused by a mutation in the NLRP7 gene.

The present invention describes the use of BMP receptor inhibitors in the prevention or treatment of hydatidiform mole diseases caused by a mutation in the familial recurrent hydatidiform mole and more particularly the NLRP7 gene.

The present invention describes BMP receptor inhibitors, more particularly BMP4 inhibitors, for use in the prevention or treatment of recurrent molar pregnancies caused by a mutation in the NLRP7 gene.

According to the invention, said BMP receptor inhibitors are preferably selected from LDN193189, Noggin, Dorsomorphin, K02288, DMH1, DMH2, LDN212854, LDN214117, ML347, and SB505124.

According to the invention, said BMP receptor inhibitors are more preferably selected from LDN193189, Noggin, Dorsomorphin, K02288 and DMH1. The most preferred BMP inhibitor according to the invention is LDN193189.

### BRIEF DESCRIPTION OF DRAWINGS

FIG. 1—Test results related to the characterization of complete mole specific iPSCs carrying the NLRP7 mutation (1A) Showing deletions on the NLRP7 gene and single base pair duplication in complete mole cells used in the study and their coordinates (1B) Colony morphology of reprogrammed cells on mouse embryonic fibroblasts (MEFs) under light microscopy. The figures have been enlarged 4X. (1C) Immunohistochemical staining (immunostaining) for pluripotency markers OCT3/4 and NANOG (1D)

Real-time polymerase chain reaction (RT-PCR) (1E) NLRP7 mRNA levels in healthy and complete mole iPSCs. Relative mRNA levels are normalized to GADPH. n=3 biological replicas. Sidak multiple comparison tests were applied following 2-way ANOVA ***P<0.005. Bars; showing±Standard Deviation. (1F) NLRP7 protein levels were measured by Immunoblotting (Western Blot Test). β-actin was used as the loading control.

FIGS. 2—(2A) PCR-based EBNA integration test on genomic DNA isolated from 3 independent clones in each genotype. In this test, the plasmid was used as the positive control. (2B) The karyotype of healthy and patient iPSC lines showing normal chromosome composition. (2C) Histological sections from in vivo teratoma forming assay showed that both iPSC cell lines generated three embryonic germ layer (endoderm, ectoderm, mesoderm).

(D) NLRP2 mRNA and protein levels in healthy and complete mole IPSCs. i) when measured by RT-qPCR ii) when measured by immunoblot assay, n=3 biological replicates. Sidak multiple comparison tests were performed following 2-way ANOVA ***P<0.001

FIG. 3—Test results showing that the NLRP7 deficiency promotes trophoblast differentiation (3A) Diagram of the trophoblast differentiation protocol (3B) Changes in colony morphology by BAP treatment. Pictures were obtained with 4× magnification. (3C) Heat map showing the expression of trophoblast markers during differentiation of healthy and complete mole iPS cells (n=2 biological replicas) (3D) Gene set enrichment analysis of the placenta module (GSEA). Genes were ranked according to the log2-fold change in gene expression of complete mole cells compared to healthy cells on day 4. (3E) Violin plot graphs showing log-transformed expression of trophoblast genes in 2C (n=2, p<0.05, Wilcoxon rank total test) (3F-3G) CDX2, HLA-G, KRT7 trophoblast markers and OCT3/4 stem cell markers immunostaining and immunoblot tests. (3F)* Percentage of immunostaining of cells for CDX2, HLA-G, KRT7 and OCT3/4. Scale bar; 10 μM. (G) Representative immunoblots for CDX2, HLA-G, KRT7 and OCT3/4.

(3H)*24-hour PGF production assessed by ELISA. (3F and 3H) n=3, following 2-way ANOVA, Sidak multiple comparison tests were applied, *p≤0.05, ***p<0.005, ****p<0.001. Rods; showing±standard deviation.

FIG. 4—(4A) Hierarchical cluster defining sample distances across the transcriptome (4B) Heat map showing the expression of trophoblast markers during the time course of differentiation of healthy cells (4C) GSEA showing the enrichment in embryonic stem cells or placenta modules. Genes were ranked according to their log2 fold change in gene expressions comparing FGF2-treated control cells with BAP-treated cells on day 4. (4D) Violin plot of pluripotency genes (n=2, p<0.05, Wilcoxon rank total test) (3E) Nuclear size of DAPI-stained cells measured by image J. n=100, Scale bar; 10 μM. ***P<0.005 Sidak multiple comparison tests were applied following 2-way ANOVA.

FIG. 5—NLRP7 deficiency does not require exogenous BMP4 exposure during trophoblast differentiation. Cells were treated with AP or FGF2 as the control. (5A) Heat map showing expression of trophoblast markers during time course of differentiation of healthy or patient iPS cells (n=2 biological replicas) (5B) Gene set enrichment analysis (Gene set enrichment analysis—GSEA) of the placental module. Genes were ranked according to the log2-fold change of gene expression in complete mole cells compared to healthy cells on day 4. (5C) Violin plot showing log-transformed expression of trophoblast genes in FIG. 5A (n=2, p<0.05, Wilcoxon rank total test) (5D) RT-qPCR for trophoblast and stem cell markers. (5E) Immunostaining for CDX2, HLA-G, KRT7 as a trophoblast markers and OCT3/4 as a pluripotency marker. Scale bar represents 10 μM (5F) Representative immunoblots for CDX2, HLA-G, KRT7 and OCT3/4. (5G) PGF production assessed by ELISA*p≤0.05, ***p<0.005, ****p<0.001 Sidak multiple comparison tests were applied following 2-way ANOVA, Bars; showing±standard deviation, n=3 biological replicas.

FIG. 6—(6A) Changes in colony morphology by AP treatment. Pictures are shown by light microscopy with 4× magnification. (6B) Hierarchical clustering defining sample distances across transcriptome (6C) Violin plot representation of pluripotent genes (n=2, p<0.05, Wilcoxon rank total test) (6D) Heat map showing expression of endoderm, mesoendoderm and mesoderm markers (z-score) (6E) Nucleus sizes of DAPI stained cells measured by ImageJ. N=100, Scale bar; 10 μM. ***P<0.005 Sidak multiple comparison tests were applied following 2-way ANOVA.

FIG. 7—NLRP7 deficiency modulates the BMP signaling pathway under AP conditions. (7A) Gene set enrichment analysis showing that the response of AP treated complete mole cells in the BMP gene set is enhanced compared to complete mole iPS cells (Gene set enrichment analysis—GSEA) Genes were ranked according to the log2-fold change in gene expression of complete mole cells compared to healthy cells on day 2. (7B) RT-qPCR showing BMP4 expression. N=3 biological replicates. (7C) BMP4 western blot analysis; SN, supernatant; WCL, whole cell lysate. (7D) Violin plot for genes responding to early BMP4 in healthy and patient cells during differentiation (p<0.05, Wilcoxon rank total test) (7E) Log-transformed expression of BMP4 target genes, GATA2 and GATA3 (7A, 7D and 7E) n=2 biological replicas, Bars; showing±standard deviation (7B and 7E) Sidak multiple comparison tests were applied following 2-way ANOVA, *p<0.05, *p<0.01, ***p<0.005, ****p<0.001 (7F) pSMAD1/5/9 and total SMAD1 western blot test

FIG. 8—BMP4 methylation status during trophoblast differentiation. The percentage of methylation at 12 different CpG regions in the BMP4 promoter and gene body was measured by pyrosequencing after AP treatment. Bars in the graphics represent mean percentage methylation, ±standard deviation. n=2 biological replicas.

FIG. 9—Preventing trophoblast differentiation in complete mole iPS cells by inhibition of the BMP signaling pathway. Cells are exposed to vehicle (DMSO) or BMP receptor inhibitor LDN193189 (100 nM) under AP conditions. (A) Changes in colony morphology by LDN193189 treatment. Pictures were obtained with 4× magnification. (B) Heat map showing the expression of trophoblast markers during differentiation of healthy and complete mole iPS cells (n=2 biological replicas) (C-D) Violin plot representation of pluripotency and trophoblast markers (n=2, ****p<0.001, Wilcoxon rank total test) (E) RT-qPCR for trophoblast and stem cell markers. Sidak multiple comparison tests were applied following 2-way ANOVA, *p<0.05, **p<0.01, ***p<0.005, ****p<0.001. Bars; showing±Standard Deviation. (F) Representative immunoblots for CDX2, HLA-G, KRT7 and OCT3/4.

FIG. 10—(10A) NLRP7 overexpression in complete mole cells by lentiviral transduction. RT-qPCR showing NLRP7 overexpression. Cells expressing GFP were used as the control. Relative mRNA levels were normalized to GAPDH. Bars in the graphic average fold change (2ΔΔCT)±standard deviation, n=3 biological replicas. (10B) BMP4 western blot analysis; SN, upper phase; WCL, whole cell lysate. (10C) CDX2, OCT3/4 immunoblot tests. B-Actin was used as the loading control. (10D) Hierarchical cluster defining sample distances across transcriptome.

### DETAILED DESCRIPTION OF THE INVENTION

The main object of the present invention is to provide prevention and/or treatment of hydatidiform mole disease, particularly familial recurrent hydatidiform mole (FRHM) disease.

The present invention provides a new method for the prevention and/or treatment of complete molar pregnancies, more particularly familial recurrent molar pregnancies, and more particularly molar pregnancies caused by a mutation in the NLRP7 gene.

The method of treatment according to the present invention involves the administration of BMP inhibitors, in particular BMP4 inhibitors, to patients with recurrent molar pregnancy or those determined to have hydatidiform mole disease.

As used herein, the term prevention and/or treatment of molar pregnancies/hydatidiform mole describes patients who have recurrent molar pregnancies and/or patients with a maternal mutation that will cause molar pregnancy to have a normal pregnancy process and have children.

Since the treatment method developed by the present invention prevents the formation of recurrent molar pregnancies and allows a healthy pregnancy period, the term prevention of the disease in accordance with the state of the art is suitable for defining the treatment method. However, further, it would be correct to say that the method provided by the present invention is also therapeutic, because the familial recurrent hydatidiform mole is considered a disease that the person carries continuously and that can cause each pregnancy to result in molar pregnancy.

In one embodiment of the present invention, BMP receptor inhibitors, more particularly BMP4 inhibitors, are used in the prevention or treatment of recurrent molar pregnancies caused by the mutations in the NLRP7 gene.

BMP (Bone morphogenetic protein) represents the bone morphogenetic protein family. In the literature, the functions of BMP family are primarily determined to induce bone and cartilage tissue formation. But also, it has a role in many important morphogenetic transmission pathways. It has a critical role particularly in early embryonic development. BMP4 is a member of the BMP family and is encoded by the BMP4 gene in humans. In the preferred embodiment of the present invention, BMP4 inhibitors are used.

For the purpose of the present invention, the inventors first investigated the function of the NLRP7 protein on complete mole pathology and found that the inadequate expression of NLRP7 differentiated a large number of stem cells into trophoblast cells, and thus caused complete mole disease.

It is already known that NLRP7 has a role in differentiation of embryonic stem cells into trophoblast cell. The inventors first obtained induced pluripotent stem cells from complete mole patients carrying the NLRP7 mutation to carry out studies on the prevention of molar pregnancies.

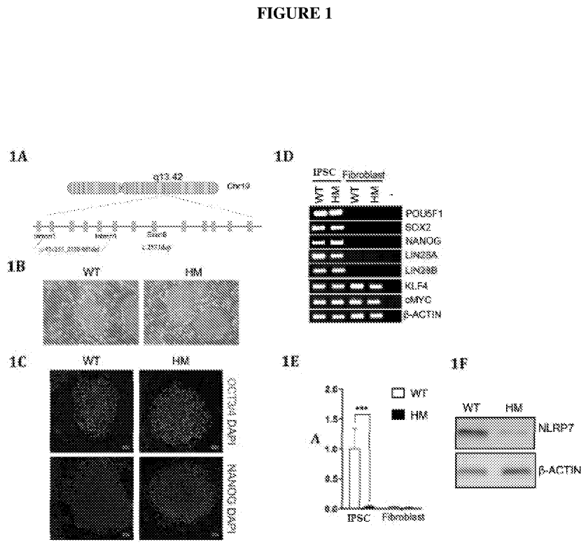

In FIG. 1A, there is a schematic view of NLRP7 gene in cells taken from a complete molar patient carrying the NLRP7 mutation used in the study. Accordingly, the mutation in the gene comprises the deletion between intron 1 and intron 5 and the c.2571 gene duplication.

Accordingly, iPSCs were obtained from fibroblasts taken from a complete mole patient with a history of 6 molar pregnancy due to NLRP7 deficiency using methods known in the state of the art (Okita et al. (2013) STEM CELLS 31(3):458-466). In order to assess the programming efficiency and pluripotency properties of the obtained iPSCs, iPSCs were also obtained from fibroblasts taken from healthy individuals. The NLRP7 status in iPSCs obtained from HM patients and healthy iPSCs were compared. The method of generating iPSCs and the details of the methods used in the experiments are described below.

The inventors did not observe a difference between the two iPSC lines in terms of reprogramming efficiency and pluripotency properties. First, they analyzed colony morphology by light microscopy (FIG. 1B). The expression of pluripotency markers OCT3/4 and NANOG markers were verified with immunofluorescence in both iPS cells, and it was proven that pluripotency markers NANOG, LIN25A, LIN28B SOX2 and POU5F1 were present in both iPS cells by real-time polymerase chain reaction (RT-PCR) (FIG. 1C-D). Thus, the inventors successfully generated iPS cells from a complete mole patient with the NLRP7 mutation.

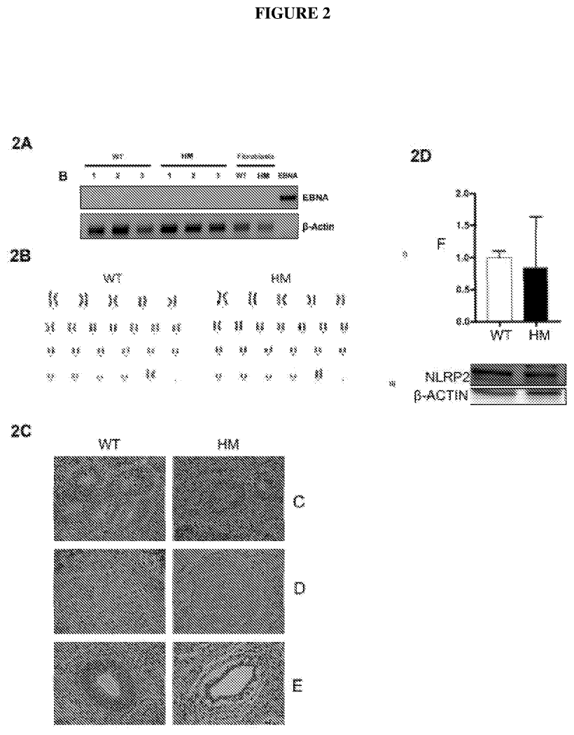

According to FIGS. 2A-2C, these iPS cells have normal karyotype. They are lack of episomal reprogramming vectors and can differentiate into three germ layers in a teratoma formation assay.

Subsequently, the inventors observed the deficiency of the NLRP7 protein in iPS cells obtained from the complete mole patient (FIG. 1E and FIG. 1F). FIG. 1E shows the comparative levels of NLRP7 expression by the quantitative real-time polymerase chain reaction analysis method. This graphic shows NLRP7 mRNA levels in healthy and sick IPS cells. FIG. 1F shows the NLRP7 protein levels in said iPS cells measured by the immunoblot test. As a result of both tests, NLRP7 expression in iPSCs obtained from a complete mole patient is too less to be detected. In light of these findings, the inventors have confirmed that NLRP7 deficiency is present in complete mole cells. At the same time, as the results can be seen in FIG. 2D, NLRP2 mRNA and protein levels are present in both healthy and complete mole cells. Results observed in NLRP7 are not valid for NLRP2, which is an adjacent gene of NLRP7.

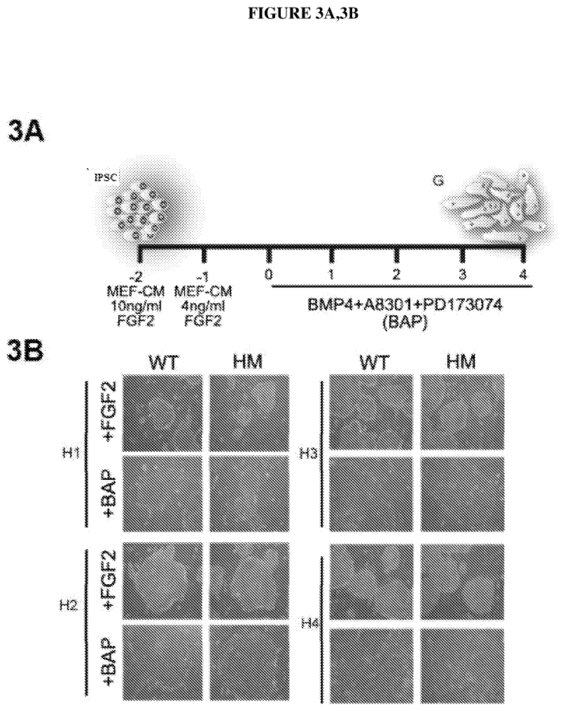

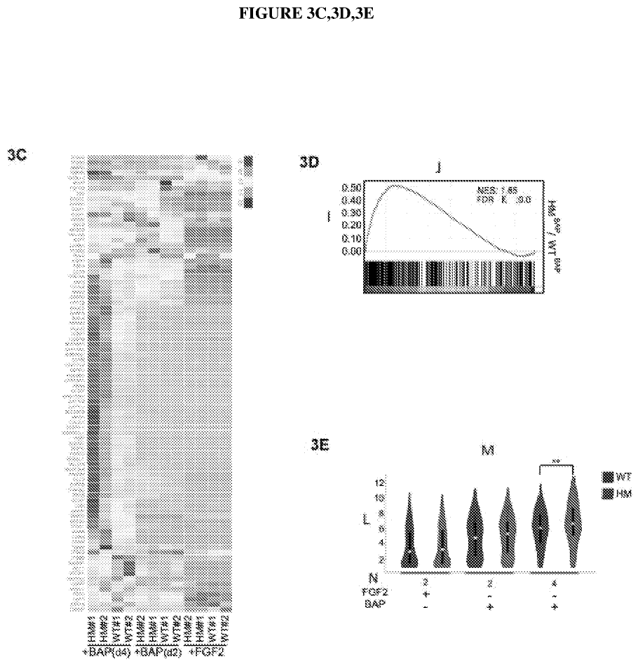

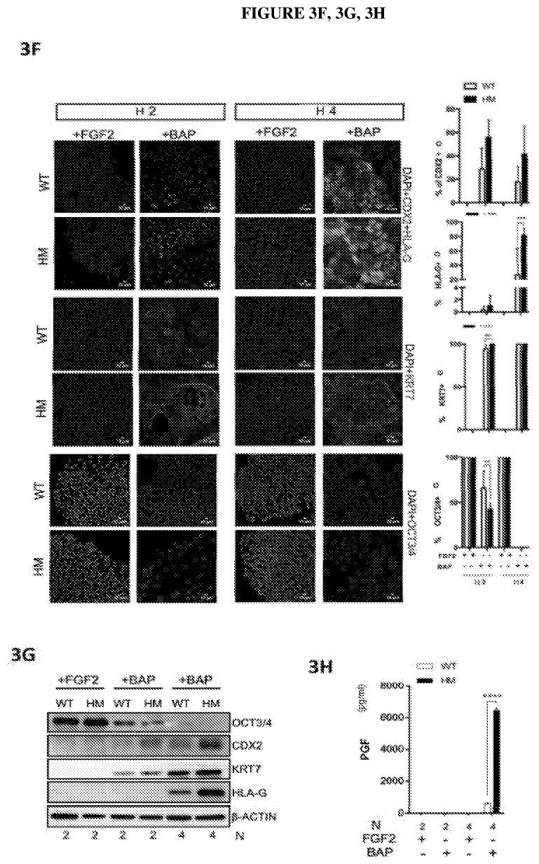

For the purpose of the present invention, the inventors obtained trophoblasts from patient-specific iPS cells and analyzed whether the pathogenesis of complete mole is present in these cells or not (FIG. 3). Trophoblast differentiation from both healthy and patient derived iPSCs were induced by BAP (BMP4+A83-01+PD173074) treatment according to the present invention.

The detailed method of obtaining trophoblast cells is shared below under the heading “Trophoblast Differentiation”. Methods of obtaining trophoblast cells are also available in the state of the art. (Amita et al. (2013) Complete and unidirectional conversion of human embryonic stem cells to trophoblast by BMP4. Proceedings of the National Academy of Sciences of the United States of America 110(13): E1212-21; Yang et al. (2015) Heightened potency of human pluripotent stem cell lines created by transient BMP4 exposure. Proceedings of the National Academy of Sciences 112(18): E2337-E2346; Yabe et al. (2016) Comparison of syncytiotrophoblast generated from human embryonic stem cells and from term placentas. Proceedings of the National Academy of Sciences 113(19): E2598-E2607)).

After exposure to BAP conditions, the change of iPS cells is detected immediately on day 2 (FIG. 3B).

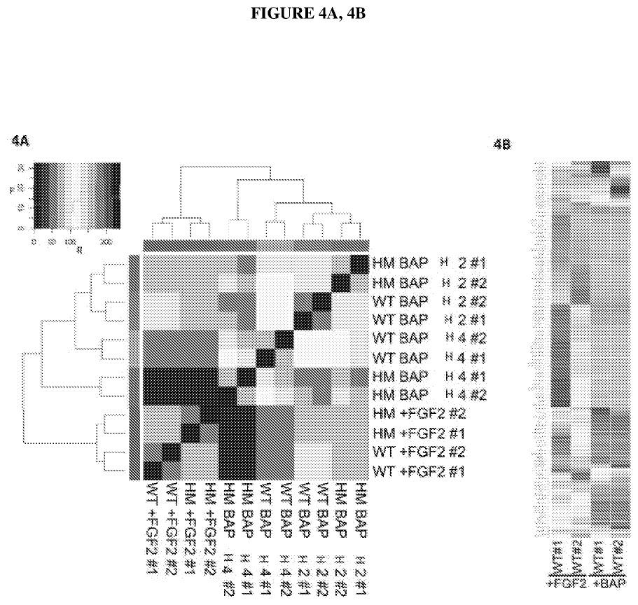

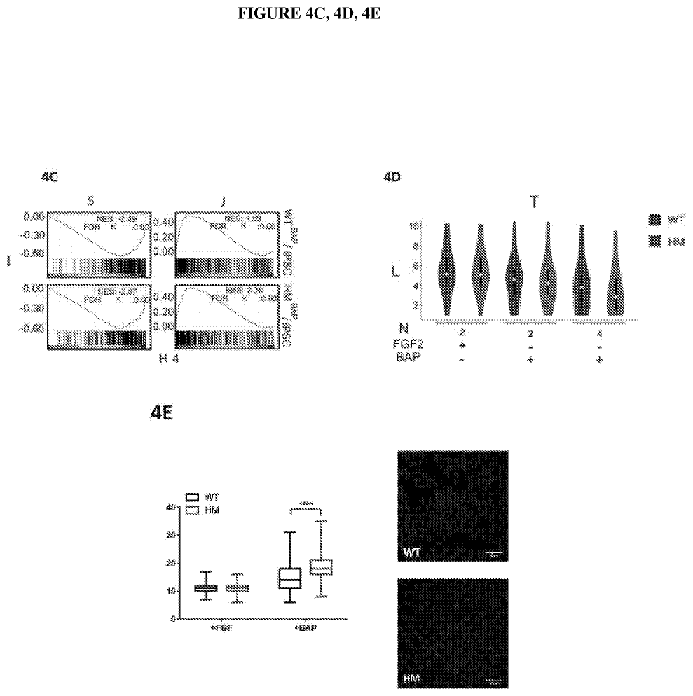

In order to detect the differentiation of cells into trophoblast cells at the whole transcriptome level, the inventors performed RNA-sequencing analysis. This analysis was performed on both healthy and patient iPS cells and its details are given in the experiments section below. The results obtained from this analysis were evaluated by hierarchical clustering and a high similarity was detected in both iPS cells. This similarity presents that NLRP7 mutations do not cause any significant transcriptional changes in the pluripotent stage. (FIG. 4A) However, transcriptomes differentiated from their iPS cells after BAP treatment. In the same way, the increase in trophoblast markers in the heat map in FIG. 4B proves the transformation of pluripotent stem cells into trophoblast type cells. The present invention is based on common trophoblast markers present in the prior art (Table 1).

TABLE 1
General trophoblast marker list.

| | | | | | | | | |
|---|---|---|---|---|---|---|---|---|
| KRT7 | LGALS16 | ITGB6 | MRGPRX1 | SLC38A3 | MAFK | LAMA1 | ITGA1 | SDF1 |
| TFAP2A | INSL4 | SLC40A1 | PSG4 | COBLL1 | PPARD | CDH1 | ITGA5 | MMP9 |
| CDX2 | GCM1 | KRT23 | TGM2 | GATA2 | DLX4 | CGB5 | ITGA6 | MMP2 |
| GATA3 | S100P | GABRP | CYP11A1 | HEY1 | PRKCH | CGB8 | ITGB1 | IGF2R |
| TFAP2C | HSD3B1 | VTCN1 | MUC15 | TRPV2 | ELF3 | CGB7 | GMNN | hCG |
| EOMES | ERVW-1 | LCP1 | ABCG2 | CEBPA | DLX3 | ERVV-1 | ITGA4 | PLAP |
| HLA-P | CDH5 | NUPR1 | SLC13A4 | LHB | EFNA1 | ERVV-2 | G11 | PTGS2 |
| PGF | HOPX | C10orf10 | PPARG | OVOL1 | MSX2 | ERVFRD-1 | Tenascin | GATA4 |
| XAGE2B | CYP19A1 | MFAP5 | CCR7 | CEBPB | GPR56 | KRT18 | FGFR3 | Z0-1 |
| ERVFRD-1 | PAPPA2 | VGLL1 | PRR5 | MXD1 | SPNS2 | ID2 | EGFR | |

In the present invention, as a result of the gene set enrichment analysis (GSEA), in complete mole cells, a decrease in embryonic stem cell specific genes and an increase in placental genes were detected. (FIG. 4C) Therefore, transcriptome of complete mole cells shows characteristics of trophoblast cells. Also, pluripotency related gene expression decreased over time in patient cells compared to healthy cells. (FIG. 3C-3E, FIG. 4E). In the present invention, the expression of commonly used trophoblast markers CDX2, HLA-G, KRT7 and pluripotency marker OCT3/4 protein was tested by immunostaining to further evaluate the differentiation efficiency of cells in trophoblast. (FIG. 3F) The details of the method is described in detail below and belongs to the state of the art. Although the early trophoblast lineage marker is increased in both BAP-treated patient and healthy iPS cells, complete mole cells show a more positive trend compared to healthy cells. For example, although CDX2 started to be positive on day 2, it tends to have more CDX2 positivity in patient cells compared to healthy cells. On the other hand, HLA-G positive cells were mostly observed where CDX2 was not present on day 4 showing later stages of differentiation. Staining for HLA-G is stronger and more uniform in complete mole cells. Further, for KRT7, the majority of cells from both groups were positive even on day 2, however, complete mole cells stained stronger than healthy cells for all evaluated time points. Consistent with this situation, OCT3/4 level decreased after 2^{nd} day of BAP treatment and complete mole cells showed greater loss of OCT3/4 than healthy cells. The inventors have noticed that in response to BAP treatment, complete mole cells become larger and more uniform than healthy cells. Accordingly, the nucleus sizes of the cells were measured with the methods present in the state of the art (with DAPI Staining). Nuclear size was measured in DAPI-stained cells. Since increasing cell size is an initial event in trophoblastic differentiation, DAPI-stained complete mole cells have significantly larger nuclei. (FIG. 4E) (Isakova and Mead, 2004; Calvert et al., 2016).

Consequently, in accordance with the immunostaining results, OCT3/4 is not detectable on day 4 of BAP treatment; CDX2, KRT7 and HLA-G increase in complete mole cells (FIG. 3G).

In the present invention, the production of placental growth factor (PGF) was also analyzed by ELISA test method (FIG. 3H). Details of the method are described below. Placental growth factor (PGF) is a placental hormone produced dominantly by functional trophoblasts during pregnancy (Khaliq et al., 1996). As a result of this analysis, it has arisen that complete mole cells produced significantly more PGF on day 4 compared to healthy cells.

When all these tests and results are combined, decreasing of pluripotency factors and activation of trophoblast lineage markers in complete mole iPS cells with NLRP7 deficiency have been observed. Trophoblast differentiation was significantly enhanced in complete mole iPS cells after BAP treatment, and the inventors have shown that these trophoblasts can successfully replicate the pathogenesis of complete mole disease.

The inventors focused on treatment after obtaining trophoblasts that successfully repeat the pathogenesis of complete mole disease caused by NLRP7 protein deficiency. First of all, the inventors analyzed the effects of NLRP7 protein expression deficiency in cells on trophoblast differentiation tendency.

Bone morphogenetic proteins (BMP) are a member of the TGF beta family. TGF-beta family is a growth factor that regulates embryogenesis. The inventors have found that the BMP4 protein and thus the signaling pathway are responsible for the phenotype observed in complete mole cells. Accordingly, it was shown that the excess trophoblast differentiation observed in complete mole cells with NLRP7 deficiency was due to the abnormal BMP4 signaling (FIG. 4B).

First of all, the inventors differentiated iPS cells obtained from the complete mole patient and healthy individual under AP conditions (A83-01+PD173074) by removing

BMP4 from the trophoblast differentiation medium and analyzed the differentiation tendency of these cells in BMP4 deficiency. Surprisingly, the inventors have found that complete mole trophoblasts differentiating under AP conditions showed similar morphology to trophoblasts under BAP conditions. Healthy cells do not differ from their undifferentiated iPSC counterparts in terms of morphology upon AP treatment.

Therefore, healthy iPS cells could not induce trophoblast differentiation in the absence of BMP4, in other words only under AP conditions.

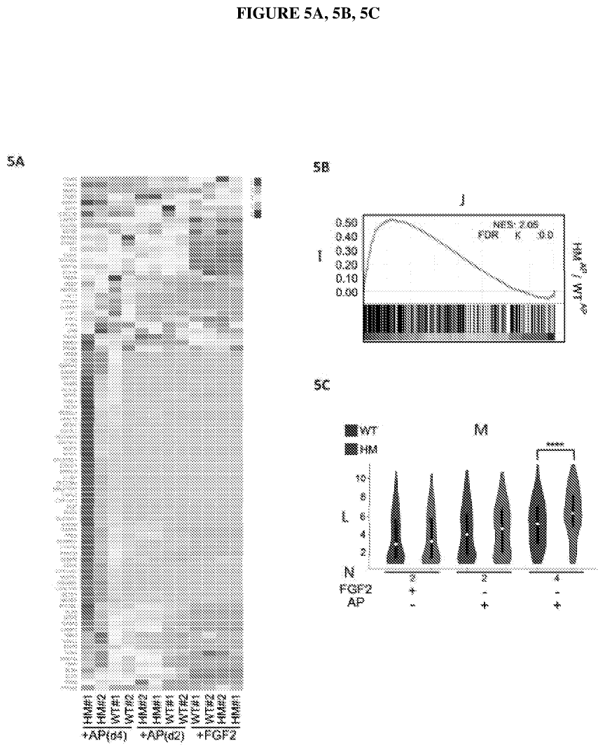

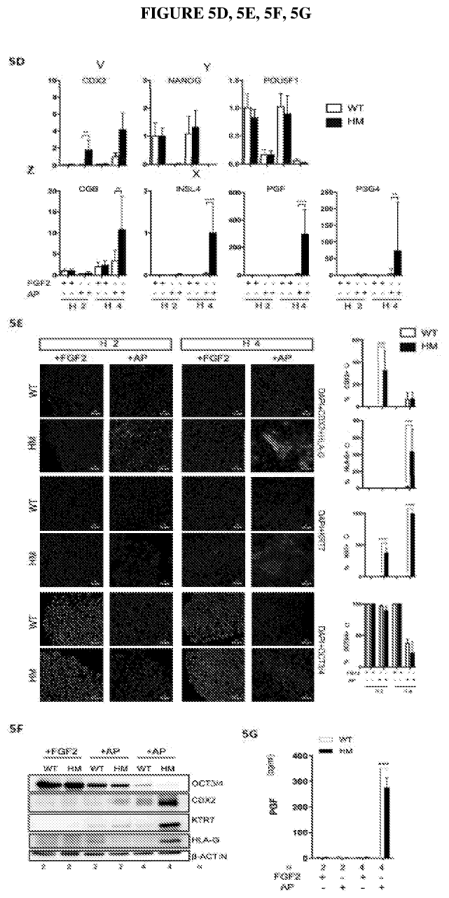

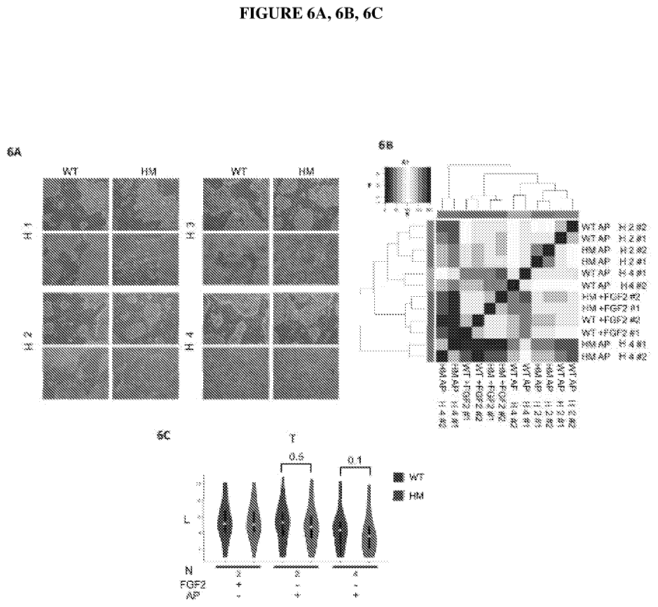

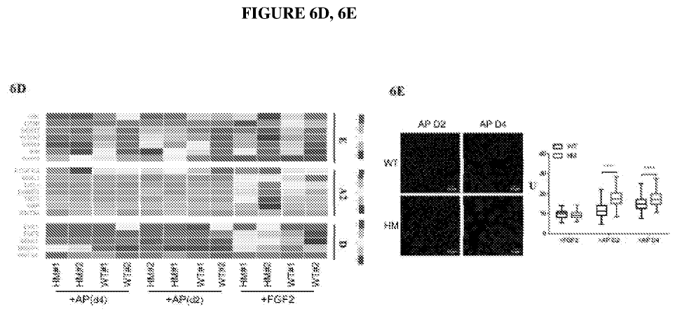

Conspicuously, according to the present invention, complete mole cells express a significant level of trophoblasts genes when treated only with AP in the absence of BMP4 (FIG. 5A). This situation is seen in the heat map in FIG. 5A showing the expression of trophoblast markers during differentiation of iPS cells into trophoblast. Further, when looked under light microscopy, the colony morphologies of BAP-treated cells were visibly identified to be similar to the morphology of complete mole cells treated with AP (FIG. 6A). In the same way, the hierarchical clustering analysis in FIG. 6B shows that the complete mole cells treated with AP are significantly different from the healthy cells treated with AP.

In accordance with the purpose of the present invention, gene set enrichment analysis was also performed on cells. The analysis is described in detail below. In the gene set enrichment analysis results in FIG. 3b, it was detected that transcripts expressed differentially in complete mole cells under AP conditions were highly associated with the placenta.

According to the present invention, while global expression of trophoblast genes was enriched in complete mole cells differentiating under AP conditions; the transcripts of pluripotency genes decreased compared to healthy cells (FIG. 5C, FIG. 6C). Also, it was confirmed by RT-qPCR that CDX2 transcripts on day 2 and PGF, INSL4, PSG on day 4 were higher in complete mole cells compared to healthy cells (FIG. 5D).

They also observed by immunostaining that CDX2 positive cells occurred in the complete mole group on day 2 and HLA-G positive cells occurred on the 4th day of AP treatment (FIG. 5E). Different from healthy cells, all of the complete mole cells stained for KRT7 on day 4. Further, as in BAP conditions, complete mole cells showed larger nuclei than healthy cells following AP treatment. Western blot analysis confirms that complete mole cells treated with AP express significantly more CDX2, KRT7 and HLA-G on day 4 compared to healthy cells (FIG. 5F). Also, OCT3/4 protein levels decrease significantly on day 4 in complete mole cells. As mentioned above, this shows that complete mole cells lose their pluripotency properties faster than healthy cells.

According to the present invention, as observed in trophoblasts obtained after BAP treatment, PGF is also secreted in complete mole trophoblasts after AP exposure. This PGF secretion is a great evidence for the trophoblastic properties of cells.

Finally, it is shown in FIG. 6E that complete mole cells have a larger nucleus compared to healthy cells, and trophoblast differentiation has been proven once again.

In accordance with these tests and their results, the inventors have demonstrated that the phenotype observed in trophoblasts differentiating under BAP conditions is present in trophoblasts differentiating under AP conditions. Observing this phenotype despite the absence of exogenous (externally given) BMP4 means that in the absence of BMP4, excess trophoblast differentiation is observed in NLRP7-deficient complete mole iPS cells. Starting from this, the inventors have demonstrated that there is a link between NLRP7 deficiency and high BMP4 signaling and obtained evidence that NLRP7 plays a role in trophoblast differentiation through BMP4 pathway.

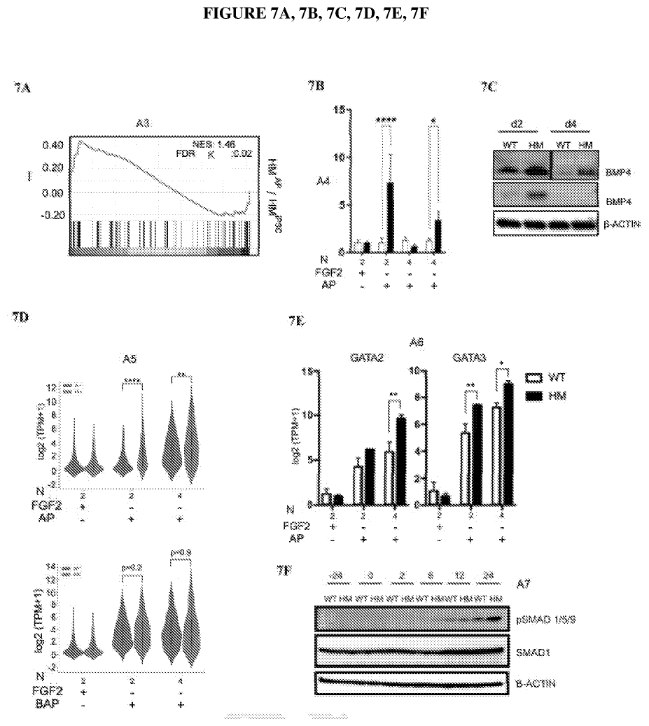

In accordance with the present invention, RNA sequencing was found to be significantly and highly enriched in the transcriptome of complete mole cells when genes associated with BMP transmission were treated with AP at the early time point (FIG. 7A). It was found by the present invention that in cultures of trophoblasts differentiated from complete mole cells under AP conditions, BMP4 mRNA levels and soluble BMP4 release were higher compared to healthy trophoblasts (FIGS. 7B and 7C).

In the present invention, in order to analyze the involvement of BMP4 signaling pathway in HM pathogenesis driven by NLRP7 mutations, the genes that responds to BMP4 was determined. This list was formed by comparing the trancriptome of BAP treated healthy cells on day 2 to that of AP treated healthy cells. The change of genes in this list in complete mole cells and healthy cells on days 2 and 4 was analyzed (FIG. 7D). The induction in expression of BMP4-responsive genes was observed in complete mole cells treated with AP (FIG. 7D). In other words, NLRP7-deficient complete mole cells were able to induce the expression of BMP4 responsive genese without being exposed to BMP4. Genes responding to BMP4 tested in the present invention are listed in Table 2. In this table, the first 200 genes whose expressions increased in healthy cells upon BAP exposure on day 2 compared to their AP treated counterparts.

TABLE 2
The list of genes responding to BMP4.

| | | | | | | |
|---|---|---|---|---|---|---|
| FOXI1 | ZNF750 | RHOXF1P3 | CASQ2 | DLX3 | C4orf19 | LINC01198 |
| TFAP2B | LIX1 | ERVE-1 | GREB1L | TFAP2A-AS1 | PDZRN4 | AP000550.3 |
| LINC00379 | TBX3 | TREH | AC243772.2 | UPK2 | MYCT1 | LINC02201 |
| SLC6A4 | DLX5 | C17orf102 | NEUROG1 | HMGCS2 | AL591806.1 | AC044839.3 |
| KCNV1 | HOXA1 | GGT3P | CRYBA1 | ACPP | ATP6V0D2 | AJ009632.2 |
| MAB21L3 | HOXC13-AS | DLX6-AS1 | HOXB6 | FYB1 | AC103957.2 | LRP2 |
| DLX6 | MEIS1 | WNT6 | FRMD7 | GGT2 | Z97192.1 | SP6 |
| DIO2 | PLCE1 | GC | GJB6 | SMIM5 | HABP2 | LINC00885 |
| SLC12A3 | LYPD2 | LINC00842 | SLC17A6 | CCR1 | MUC15 | WLS |
| BARX2 | AL138686.1 | SCML4 | SPAM1 | HAPLN1 | EPAS1 | AC011298.1 |
| OR7E91P | DIO3OS | DDC | TMPRSS4 | CDH10 | FAM89A | AC090673.1 |
| ARHGEF38 | LCMT1-AS2 | TLR7 | GATA2-AS1 | AMER2 | C10orf71 | MSX2 |
| HAND1 | LINC02458 | CDX2 | GATA3 | TRIML1 | TNFSF8 | ACADL |
| HOXB2 | HLA-DRB6 | GATA3-AS1 | TRIM55 | HOXB5 | ISL1 | AC108752.1 |
| NKX2-3 | KCNJ13 | MEIS2 | MGAM2 | PPARG | VIT | ZBTB7C |
| NKX2-6 | FUT8-AS1 | AC092118.1 | AL035661.1 | AC099676.1 | KANK4 | AC020656.2 |
| AL109613.1 | AL355499.2 | PRRT1B | CCR7 | AC005041.3 | ATP6V1B1 | LINC00518 |
| TFAP2A | MEIS1-AS3 | FOXF2 | ATP6V1C2 | TNNT3 | TMEM132E | AP001207.3 |
| MSX1 | SLC8A1 | CLIC5 | SULT2A1 | MS4A4A | GATA2 | COL19A1 |
| TPRXL | PSCA | HOTAIRM1 | FOXE3 | RARB | PRTG | |
| LRP1B | AC127521.1 | P2RY6 | SGCZ | HOXB3 | AC130456.2 | |
| AC110619.1 | HOXC13 | FGB | AL645608.1 | FYB2 | ATP12A | |
| TBX2 | SH3TC1 | AC012501.2 | LMO1 | AC244453.2 | OLFML1 | |
| AC116667.1 | VGLL1 | DLC1 | ERP27 | CSF3R | SEMA6D | |
| SPNS2 | CHI3L2 | HNRNPA1P33 | ENPEP | SPATA41 | PRAMEF12 | |
| TCAF2 | LHX5-AS1 | FGG | AC129926.1 | AC012574.1 | TTR | |
| AL390294.1 | GAS1RR | AL353732.1 | ARHGAP27P2 | LMOD2 | AL021392.1 | |
| KCNA4 | AC116612.1 | PLA2G12B | DIRAS3 | SCN7A | GLRA3 | |
| VTCN1 | PKDCC | CNTN4 | PKP1 | AC005863.1 | AL355499.1 | |
| AC010478.1 | KRT80 | LHFPL6 | FEZF2 | CES5AP1 | TRIM17 | |

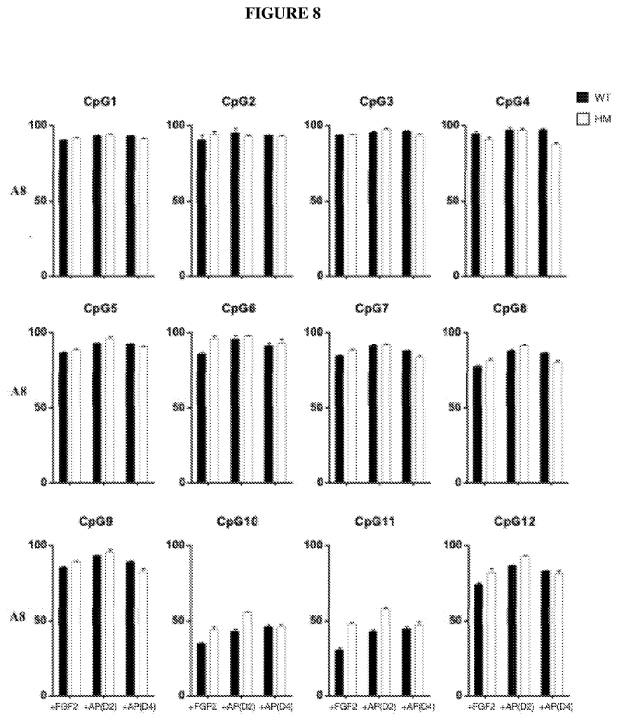

GATA-2 and GATA-3 are important transcription factors for trophoblast differentiation during embryogenesis. According to the q-RT-PCR analysis of the results in FIG. 8E, it was found that the expression of GATA-2 and GATA-3 was significantly higher in complete mole cells. Thus, it has been seen that the BMP4 signaling pathway is active in complete mole cells with NLRP7 mutations under AP conditions where BMP4 is not given externally.

Finally, it was determined by western blot analysis that the phosphorylation of pSMAD1/5/9 increased significantly during the trophoblast differentiation of complete mole cells under AP conditions implying an active BMP4 signaling (FIG. 7F). This situation shows that the underlying mechanism of increased trophoblast differentiation in complete mole cells is an overactive BMP4 signaling pathway.

In accordance with all these tests, the inventors surprisingly found that there is an interrelation between NLRP7 and BMP4 and that the excessive trophoblast differentiation observed in complete mole cells deficient in NLRP7 is caused by the abnormal BMP4 signaling pathway. Also, the inventors found that NLRP7 regulates BMP4 pathway that is critical in early embryonic development.

In accordance with the purpose of the present invention, the inventors stopped the excess trophoblast differentiation of complete mole cells by inhibiting the BMP4 signaling pathway.

In the treatment in accordance with the present invention, BMP receptor inhibitors, preferably BMP4 receptor inhibitors are used to reduce trophoblast differentiation.

The present invention describes the use of BMP receptor inhibitors in the prevention or treatment of molar pregnancies, more particularly familial recurrent hydatidiform mole, and more particularly hydatidiform mole diseases caused by mutations in the NLRP7 gene.

According to the present invention, molar pregnancy is particularly a familial recurrent hydatidiform mole (FRHM) and more particularly complete mole pregnancy caused by mutations in the NLRP7 gene.

BMP receptor inhibitors according to the present invention are preferably selected from a group consisting of LDN193189 (DM-3189), Noggin, Dorsomorphin, K02288, DMH1, DMH2, LDN212854, LDN214117, ML347, SB505124 and derivatives.

Said BMP receptor according to the invention inhibitors are preferably selected from LDN193189, Noggin, Dorsomorphin, K02288 and DMH1 and most preferred is Noggin or LDN193189. The formula for the LDN193189 molecule is below (Formula 1):

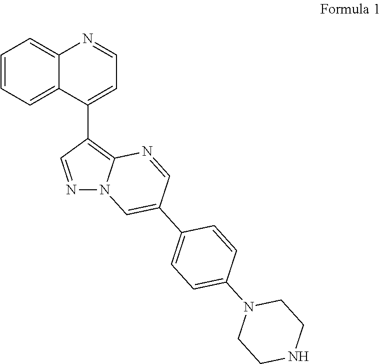 [SMILES: c1ccc2c(-c3cnn4cc(-c5ccc(N6CCNCC6)cc5)cnc34)ccnc2c1]

The BMP receptor inhibitor according to the present invention is preferably BMP4 inhibitor.

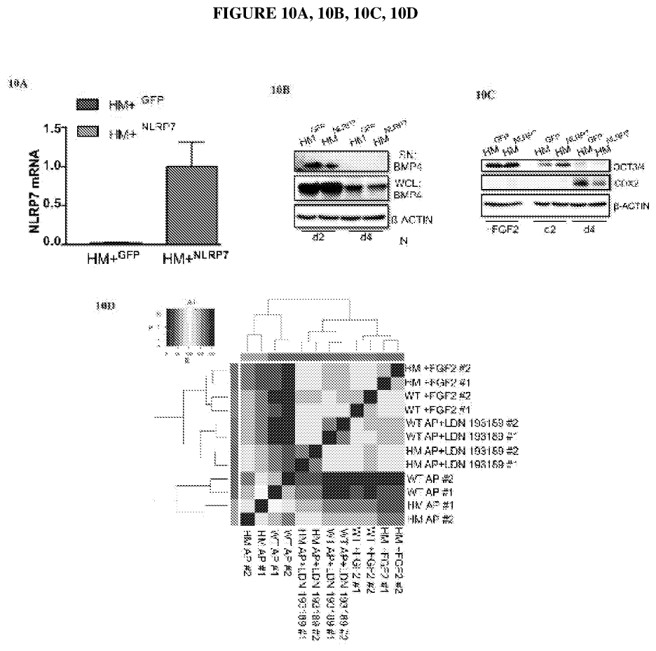

In accordance the purpose of the present invention, the inventors applied it to the nutrition fluid of cells under LDN193189 AP conditions to test whether BMP receptor inhibitors can attenuate trophoblast differentiation or not. As a result, complete mole cells exposed to LDN193189 proved that they showed a morphological phenotype similar to healthy cells and showed that BMP receptor inhibitors would prevent complete molar pregnancies. (FIG. 10A)

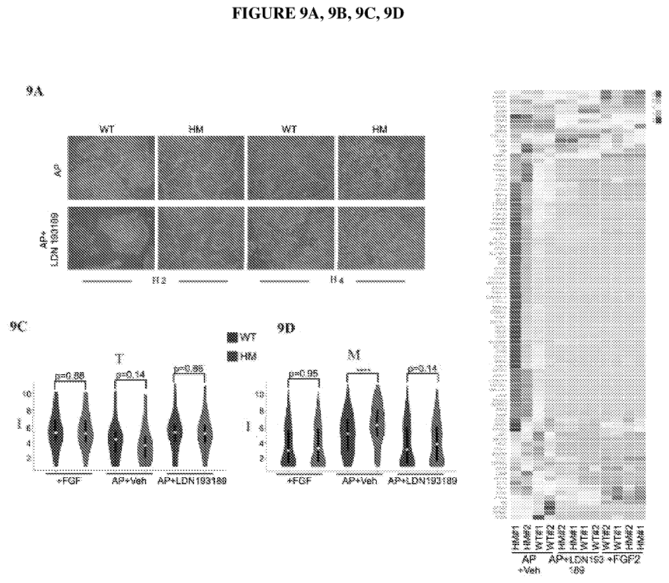

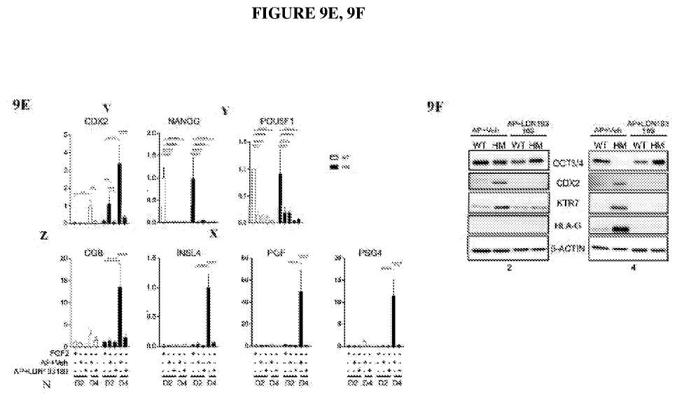

As shown in FIG. 9A, colony morphology was analyzed by light microscope. Here, the morphological similarity is very high. The temperature map in FIG. 9B also clearly shows that BMP pathway inhibition (suppression by inhibitor) inhibits trophoblast differentiation of complete mole cells. Global expression of trophoblast markers also decreased in response to LDN193189; pluripotency markers remained highly expressed (FIGS. 9C and 9D). In addition, the decrease in trophoblast gene expression following BMP pathway inhibition by qRT-PCR was also confirmed by the inventors (FIG. 9E). According to transcriptomic analyses, BMP pathway inhibition stopped CDX2, KRT7, HLA-G increase in expression (upregulation) and prevented decrease in expression (downregulation) of OCT3/4 in complete mole cells (FIG. 9F). Generally, these results show that the tendency to lose pluripotency properties and to rapidly differentiate into trophoblasts in complete mole cells is associated with abnormal BMP signaling.

The inventors also showed by hierarchical clustering analysis that at all transcriptome level, cells treated with LDN193189 were clustered with non-differentiated iPS cells (FIG. 10D).

The suppression of excessive trophoblast differentiation by the BMP receptor inhibitor LDN193189 shows that the BMP4 pathway plays a role in the trophoblast differentiation tendency caused by NLRP7 deficiency in complete mole cells.

In the preferred embodiment of the invention, at least one BMP inhibitor according to the invention is added to the nutrient fluid of the fertilized ovum prepared for in vitro fertilization (IVF) in the treatment performed to prevent a new molar pregnancy of the patient with recurrent mole pregnancy and the fertilized ovum is grown in this nutrient medium at the development stage before it is introduced into the uterus.

Experiments

Using non-integrated episomal plasmids, fibroblasts from the complete mole patient and healthy individual were reprogrammed.

Material and Method

Primary Culture from Human Skin Biopsy

Skin samples from a 30-year-old female patient and a 40-year-old healthy volunteer were transferred to DMEM medium in ice (GIBCO®). The samples were washed with PBS and cut into small pieces with a surgical lance. The smallest pieces were placed in 6-well plates and closed with 22 mm glass lid to be stabilized within 4° C. complete DMEM (DMEM supplemented with 10% FBS), 2 mM L-Glutamine, 1× MEM non-essential amino acids, 100 U/ml penicillin and 100 μg/ml streptomycin. The medium is changed every 3-4 days. 10% DMSO, 10% FBS, 80% complete DMEM are used to freeze the cells.

IPSC Formation Specific to Patient

In the state of the art, patient-specific IPSCs are formed with existing episomal transfection. (Okita et al. (2013) STEM CELLS 31 (3): 458-466). The day before episomal transfection of the reprogramming vectors, primary fibroblast cells were seeded in the absence of 3×105 in 6-well plates and incubated overnight at 37° C. in 5% CO2. Each plasmid was transfected 1 μg from pCXLE-Oct3/4-shp53, pCXLE-SK, pCXLE-UL, pCXWB-EBNA and CXLE-eGFP, pCXWB-EBNA, 1400V, 20 ms were used as the transfection control through electroporation with 2 pulse by NeonQR Transfection System (Thermo Scientific). Six days after transfection, mitomycin-c (Sigma) treated MEFs were seeded on 6-well plates coated with 0.2% gelatin (Sigma). The next day (day 7) reprogrammed cells were harvested and transferred to plates containing MEF. Then, the medium was replaced every other day with hES medium containing 10 ng/mL of FGF2 (Peprotech).

Teratoma Formation Test

Three confluent 10 cm plates were separated using regular passage protocols and resuspended in 50% Matrigel (Corning) and 50% cold DMEM medium supplemented with 10% FBS, 2 mM L-Glutamine, 100 U/ml penicillin and 100 μg/ml streptomycin and kept on ice. The mixture was administered to three SCID mice by intramuscular injection under anesthesia. Anesthetized mice were sacrificed by an IACUC approved method after injection for 6-8 weeks and the teratoma was cut and separated. The teratome was fixed in 10% formalin. Histopathological staining and examination was performed.

Trophoblast Differentiation

Trophoblast cells were formed from the obtained IPSCs under BAP or AP conditions. IPS cells were routinely kept on MEFs with hES medium (10 ng/mL FGF2). For trophoblast differentiation, 2.4×10⁴ cells per square centimeter were seeded on matrigel-coated plates with conditioned hES medium through a monolayer of mitomycin-C treated MEF nutrient cells (MEF-CM) containing FGF2 (10 ng/mL). Next day, the medium was changed as MEF-CM containing 4 ng/mL FGF2. Next day, the medium was changed as BMP4 (10 ng/mL) containing unconditioned (BAP) hESC basal medium with MEF nutrient cells (RD Systems), ALK4/5/7 inhibitor, A83-01 (1 μM) (Tocris) and FGF2-signal inhibitor PD173074 (0.1μ) (Sigma) (22, 27, 28). Control cultures were grown in the presence of FGF2 and in the absence of BAP. The medium was refreshed daily.

Quantitative Real-Time Polymerase Chain Reaction

Total RNA was extracted using the Direct-zol RNA Isolation Kit (Zymogen) and cDNA was synthesized using the Sensifast cDNA synthesis kit (Bioline) as described by the manufacturer. Sequences of primers used in gene expression were synthesized by Macrogen as listed in table 3. qRT-PCR was performed on Exicycler™ 96 (Bioneer) using SensiFAST™ SYBRQR No-ROX Kit (Bioline). qRT-PCR results were analysed by the Ct method for relative amounts by taking GAPDH or HPRT as internal controls.

TABLE 3
List of qRT-PCR primers

| qRT-PCR | | |
|---|---|---|
| PRIMER | Forward | Backward |
| BMP4 | 5′TCCTGGTAACCGAATGCTGA | 5′CCTGAATCTCGGCGACTTTT |
| CDX2 | 5′GCCAAGTGAAAACCAGGACG | 5′TCCTCCGGATGGTGATGTAG |
| CGB | 5′GTCAACACCACCATGTGTGC | 5′GGTAGTTGCACACCACCTGA |
| GAPDH | 5′GGAGCGAGATCCCTCCAAAAT | 5′GGCTGTTGTCATACTTCTCATGG |
| HLA-P | 5′CTCTCAGGCTGCAATGTGAA | 5′CATGAGGAAGAGGGTCATGG |
| INSL-4 | 5′CCCCATGCCTGAGAAGACAT | 5′GTTGTTGGAGGTTGACACCATT |
| NANOG | 5′CATGAGTGTGGATCCAGCTTG | 5′CCTGAATAAGCAGATCCATGG |
| NLRP2 | 5′GCTGCTGTGTTGGTTGTCAG | 5′GCAGTTCCAAAGCACCAAGG |
| NLRP7 | 5′TAAGGAATGCGACTGTGAACATC | 5′TGCTAACTCCGAGTCTTCTTCT |
| PGF | 5′TCCTACGTGGAGCTGACGTT | 5′CACCTTTCCGGCTTCATCTTC |
| POU5F1 | 5′GGCTCGAGAAGGATGTGGT | 5′GCCTCAAAATCCTCTCGTTG |
| PSG4 | 5′CCAGGGTAAAGCGACCCATT | 5′AAGAATATTGTGCCCGTGGGTT |

EBNA Integration Test

Genomic DNA was isolated from IPSC cells by means of a commercial kit (MACHEREY-NAGEL). PCR testing was performed using the following primers with 50 ng of genomic DNA as the model per reaction:

EBNA-Fwd:
AGGGCCAAGACATAGAGATG,
EBNA-Rev:
GCCAATGCAACTTGGACGTT,
GAPDH-Fwd:
ATCACCATCTTCCAGGAGCGA
GADPH-Rev:
TTCTCCATGGTGGTGAAGACG

PCR products were sequenced by Macrogen Inc (Korea).

Immunofluorescence Staining (Immunohistochemical Staining)

Cells were fixed in 4% paraformaldehyde (PFA; Sigma) for 30 minutes at room temperature and washed three times with phosphate buffered saline (PBS). Subsequently, cells were permeabilized with 0.2% TritonX-100 (Sigma) for 20 minutes at room temperature and washed three times with PBS. Cells were blocked in 3% BSA (Applichem) and 5% donkey serum (Merck) in PBS for 2 hours at room temperature. Immune labelling was performed at 4° C. overnight by using CDX2 (EPR2764Y, ABCAM) (1:250), KRT7 (M7018, DAKO) (1:100), OCT3/4 (sc-5279, SantaCruz) (1:100), NANOG (AB21624, ABCAM) (1:100), Mice IgG (0.4 μg/mL). Next day, cells were washed 3 times with 1× PBS and incubated with appropriate secondary antibodies conjugated with Alexa-Flour 488, 555 or 568 for 3 hours at +4° C. in the dark. Cells were washed 3 more times with 1× PBS. Images were obtained by using a confocal microscope (Leica TCS SP8, USA) and processed through ImageJ.

Immunoblot Test (Western Blot Test)

Cells were harvested in RIPA lysis buffer (150 mM NaCl, 1% NP40, 0.5% sodium deoxycholate, 0.1% SDS, 50 mM Tris pH 7.4) supplied with protease and phosphatase inhibitors (Roche, Switzerland). TCA-acetone precipitation was performed for the supernatants. Protein samples were applied to the SDS gel. Semi-dry transfer was performed by using Blotting papers (Sigma-Aldrich, USA) and PVDF membrane (Millipore, Ireland). After blocking with 5% non-fat dry milk, membranes were incubated overnight with primary antibodies (1:1000) at +4° C. Next day, the membrane was incubated with secondary antibodies connected with 1:2000 HRP according to the host origin of the primary antibody. Between all steps, membranes were washed 3 times with TBS-T. Immunoblot membranes were visualized with the Syngene documentation system by using ECL HRP Substrate (Advansta, USA).

PGF Determination in Complete Mole and Healthy IPSCs after BAP Treatment—Immunoassay (ELISA)

PGF was measured by using the Human PIGF ELISA (DPG00; RD Systems) according to the protocol of the manufacturer. After dilution to the appropriate sample and standard concentration, it was transferred onto a 96-well plate and incubated for 2 hours at room temperature. The wells were washed four times with washing buffer and conjugated antibody was added to the plate and incubated at room temperature for 2 hours. Substrate Solution (1:1 Color Reagent A (H2O2): Color Reagent B (Tetramethylbenzidine)) was incubated in the dark at room temperature for 30 minutes. Finally, stop Solution (2M H2SO2) was added and the reaction was stopped by mixing and optical density was measured at 450 and 570 nm (VersaMax, Molecular Devices, USA).

RNA Sequencing Analysis

Total RNA was extracted by using the Direct-zol RNA Isolation Kit (Zymogen) according to the protocol of the manufacturer (n=2 in all cases). Library preparation procedure by using Truseq strand mRNA LT Sample Preparation Kit (Illumina) and RNA sequencing procedure by using Hiseq2500 (Illumina) were performed by Macrogen Inc. (Korea). RNA sequence data were processed and interpreted with the Genialis visual computing platform (www.genialis.com). Briefly, RNA sequence readings were subjected to correction (BBDuk), mapping (STAR) and expression quantitation (featureCounts) procedures, respectively. The readings were mapped to Homo sapiens GRCh38 (Ensembl, version 92, ERCC). Differential gene expression analyses were performed by using DESeq2 (Love, Huber and Anders, 2014). The low-expressed genes were filtered from the differential expression analysis input matrix. Expression level (TPM, transcripts per million kilo of base) was determined by Cufflinks (http://cufflinks.cbcb.umd.edu). Temperature maps were formed with the Genialis platform according to scaled in rows expression by using Z-score based on TPM values of specific gene sets of general trophoblasts, CTs, EVTs or STs indicated in the literature. (Appendix Table 1, 2) (Yang et al., 2015; Lee et al., 2016; Yabe et al., 2016; Okae et al., 2018; Vento-Tormo et al., 2018). Benporath-ES1 gene set was used as pluripotans markers (Ben-Porath et al., 2008). On day 2, the first 200 genes differential upregulated in WTBAP cells compared to WTAP cells are assigned as early BMP4 sensitive genes (Appendix Table 3). Hierarchical clustering was performed by using R, DEseq2 package to show sample distances. Violin graphics were formed by using Python 3.7.2 with the Seaborn library. All of the RNA sequence data can be accessed with the GEO code GSE125592.

Gene Set Enrichment Analysis.

The pre-ranked differentially expressed gene lists were applied to GSEA by using default settings (http://software.broadinstitute.org). BENPORATH ES 1 for identification of enrichments in pluripotans genes; placental genes, Module 38, and GO response of the BMP pathway to BMP modules were evaluated.

### DESCRIPTION OF THE REFERENCES IN FIGURES FIG. 1B:

WT: Health cell

HM: Hydatidiform mole sick cell

FIG. 1E:

A: NLRP7 Relative Expression

FIG. 2A:

B: Colony Number

FIG. 2C:

C: Ectoderm

D: Mesoderm

E: Endoderm

FIG. 2D:

F: NLRP2 Relative Expression

FIG. 3A:

G: Trophoblast-type cells

FIG. 3B:

H: Day

FIG. 3D:

I: Enrichment score J: Placenta module

K: q-value

FIG. 3E:

L: Log2 (TPM+1) expression M: Trophoblast genes

N: Time (day)

FIG. 3F:

O: cells/area

FIG. 4A:

P: Number R: Value

FIG. 4C:

S: ESC module

FIG. 4D:

T: Pluripotency genes

FIG. 4E:

U: Nucleus diameter (μm)

FIG. 5D:

V: Early trophoblast marker

Y: Sternness marker Z: Relative mRNA expressions X: Late trophoblast markers

FIG. 6B:

A1: Chroma key and histogram

FIG. 6D:

A2: Mesoendoderm

FIG. 7A:

A3: Respond to BMP module

FIG. 7B:

A4: BMP4 Relative Expression

FIG. 7D:

A5: Early BMP4 responsive genes

FIG. 7E:

A6: BMP4 target genes

FIG. 7F:

A7: Time (hour)

FIG. 8:

A8: % Methylation

1-8. (canceled)

9. A method of using a BMP receptor inhibitor or a pharmaceutically acceptable derivative thereof in the prevention or treatment of molar pregnancies.

10. The method of claim 9, wherein molar pregnancy is a complete molar pregnancy.

11. The method of claim 9, wherein molar pregnancy is familial recurrent hydatidiform mole.

12. The method of claim 9, wherein said molar pregnancy is caused by a mutation in the NLRP7 gene.

13. The method of claim 9, wherein said inhibitor is BMP4 receptor inhibitor.

14. The method of claim 9, wherein said inhibitor is selected from the group consisting of LDN193189, Noggin, Dorsomorphin, K02288, DMH1, DMH2, LDN212854, LDN214117, ML347, SB505124 and derivatives thereof.

15. The method of claim 9, wherein said inhibitor is selected from the group consisting of LDN193189, Noggin, Dorsomorphin, K02288, DMH1 and derivatives thereof.

16. The method of claim 9, wherein said inhibitor is LDN193189.
